(19) United States
(12) Patent Application Publication
(10) Pub. No.: US 2013/0211290 A1
(43) Pub. Date: Aug. 15, 2013

(54) OUTSOLE SHEET FOR GAIT DIAGNOSIS, AND FOOTWEAR SYSTEM FOR GAIT DIAGNOSIS AND SERVICE SYSTEM FOR GAIT POSTURE DIAGNOSIS USING SAME

(71) Applicant: Jin-Wook Lee, Seoul (KR)

(72) Inventor: Jin-Wook Lee, Seoul (KR)

(21) Appl. No.: 13/882,408

(22) PCT Filed: Oct. 27, 2011
(86) PCT No.: PCT/KR2011/008089

§ 371 Date: Apr. 29, 2013

(30) Foreign Application Priority Data
Nov. 2, 2010 (KR) 10-2010-0107913

(51) Int. Cl. A61B 5/00; A61B 5/103

(52) U.S. Cl. CPC A61B 5/6807; A61B 5/1038; A61B 5/0004; A61B 5/0026; A61B 5/742; A61B 5/0015
USPC 600/592

(57) ABSTRACT

A shoes system for walk diagnosis using an insole sheet for walk diagnosis includes shoes for walk diagnosis including a PCB installation groove having a stepped groove at one upper side of an insole to mount a system for detecting a gait, a cover attached onto the insole, a pressure detection sheet placed on the cover to generate a switching signal corresponding to a pressure applied by a walker, and a circuit unit that is installed in the PCB installation groove, is connected to a connecting unit of the pressure detection sheet, accumulates and manages signals detected from the connecting part for each time slot, supplies wireless power to the system, and wirelessly transmits data corresponding to walk information, and a charging stage including a mounting panel for mounting the shoes for walk diagnosis.

### CROSS-REFERENCE TO RELATED APPLICATION

This application claims priority under 35 USC §119 to Korean Patent Applications No. 10-2010-0107913, filed on Nov. 2, 2010 in the Korean Intellectual Property Office (KIPO), the contents of which are incorporated herein in its entirety by reference.

### BACKGROUND

1. Technical Field

The present invention relates to a gait diagnosis system, and more particularly, to an insole sheet having a simple structure for recognizing and analyzing a gait of a user and for widening the scope of application of the system, shoes for diagnosing a gait using the same, and a gait diagnosis service system.

2. Description of the Related Art

Walking is one of the characteristics of a human being. People walk in everyday life and sometimes walk long distances intentionally for health. However, people have different gaits. People have different body postures, different walking speeds, and different foot movement paths when they walk.

In general, the sole of a human foot has an arcuate structure. This arcuate structure serves as a lever for supporting the weight of a body and serves as a spring for walking or running. However, when a person stands or walks for a long time, a flat foot not having such an arcuate structure easily causes fatigue or even serious pain.

In particular, a flat foot of a juvenile often causes an ankle wrench or harmfully affects a protruding bone under an anklebone, bringing about abnormal growth of a frame. Moreover, in a serious case, the balance of the shinbone (i.e., leg bone) is broken, causing various serious diseases. In particular, a flat foot of a child is more serious and causes abnormality of a joint of a foot which may develop into arthritis. Therefore, it is needed to pay particular attention, and it is required to prevent and correct a flat foot.

Accordingly, it is very important for both children and adults to walk rightly. Wrong walk may harmfully affect the growth and body shapes of children and juveniles. Even adults may experience health problems due to wrong walk.

FIG. 7 is a diagram illustrating conventional shoes for diagnosing a walker.

Referring to FIG. 7, Korean Patent Application Laid-open Publication No. 10-2007-0100592, entitled “Gait Analysis System”, discloses a system for analyzing a gait of a walker. This system is described in detail as below.

As illustrated in FIG. 7, a gait element generation unit 100 is provided with various sensors 11, 12, and 20, a moving path estimation module 110, a walk detection module 120, an absolute direction detection module 130, and a pressure change detection module 140. The sensors for variously measuring a walk motion include an inertial sensor 11, a geomagnetic sensor 12, and a pressure sensor 20.

The moving path estimation module 110 calculates a moving path of a foot from acceleration and angular velocity measured in the inertial sensor 11 by using a navigation equation of a strap-down inertial navigation system (SDINS). The moving path estimation module 110 includes an SDINS unit, a foot landing determination unit, and a foot landing correction unit. The SDINS unit calculates a three-dimensional moving path (i.e. position, velocity, and posture) of a foot from tri-axial acceleration and tri-axial angular velocity measured in the inertial sensor 11 through a known navigation equation. Here, due to various factors, the calculated moving path includes an error. Therefore, error compensation is performed to obtain a correct moving path. For the error compensation, known zero velocity compensation (ZVC) is performed using the fact that the speed of a foot is 0 at a moment of foot landing.

The moment of foot landing may be detected by using a detection value of the pressure sensor 20 in the foot landing determination unit, and the error compensation is performed by designing a known Kalman filter in the foot landing correction unit.

The walk detection module 120 determines whether a user walks by using the inertial sensor 11 and calculates the number of steps per unit time to thereby obtain a walk frequency. The walk detection module 120 inputs a standard deviation of acceleration data and the walk frequency to a stride estimation model so as to calculate a stride.

The above-described conventional gait analysis system may variously measure a walk motion of a user to obtain gait elements and may compare the gait elements with those of a right gait to provide a result of the comparison to the user. By virtue of this information, the user may easily detect problems of a gait of the user and may find a way of improvement.

In particular, data on a gait desired by a user may be stored so as to provide a customized service by using the stored gait as a reference gait. Further, the above-described system may be utilized as an expert system that provides, to a user, health problems caused by a wrong gait. In additional, the system may be directly implemented in various mobile terminals such as personal navigation devices, cell phones, and PDAs, or may be used in association with the mobile terminals.

The above-described gait analysis system is equipped with various multiple sensors and extracts a gait of a user by analyzing signals detected from the sensors. There are products having structures similar to the structure of the system, such as NIKE Plus, “Micoach” of Adidas, “The electronic Pedometer” of PUMA, “Cairun” of Aison, “GPS Smart Shoes” of GTXC, and “Smart Shoe” of Apple which are commercialized or are being developed. These products are equipped with sensors and recognize a posture state of a walker, and then transmit the posture state with a certain communication device.

However, a sensor that is installed in the inside, e.g. the insole, of a shoe for analyzing a gait may provide correct data. However, due to a high price of such a sensor, seniors or children may not afford to purchase such shoes. In addition, since many components are installed in a shoe, data should be managed or the system should be charged after a lapse of certain time, degrading convenience of use. This limitation hinders expansion of the market of such shoes.

### SUMMARY

Some example embodiments provide an insole for walk diagnosis having a simple sensor for detecting a walk to remarkably reduce the cost of production and widely distribute shoes for walk diagnosis or analysis, and shoes for walk diagnosis using the same.

Some example embodiments provide a walk diagnosis system using shoes for walk diagnosis for easily managing walk diagnosis information by providing accumulated walk diagnosis information to online networks based on short-range wireless communication when the shoes for walk diagnosis are taken off at a certain position.

Some example embodiments provide a walk diagnosis system using shoes for walk diagnosis for continuously managing the heath of the old and the infirm by continuously accumulating walk diagnosis information of the old and the infirm, diagnosing the health on the basis of the information, and notifying results of the diagnosis to local governments or family doctors.

According to some example embodiments, an insole sheet for walk diagnosis for use in shoes for collecting walk information of a walker may be provided. Here, a pressure detection sheet having a film shape may be formed to have a structure corresponding to the insole, a plurality of switching units may be arranged on a surface of the pressure detection sheet, a circuit pattern for recognizing an electrical connection between the plurality of switching units may be formed, and a connecting unit may be extended to an inner side of the pressure detection sheet to aggregate the circuit pattern.

In example embodiments, the pressure detection sheet may include a first sheet contacting an inner bottom of the shoe and a second sheet contacting a body of a user. Here, a contact switch and a pattern may be formed between the first and second sheets at a position corresponding to the switching unit, and a bonding sheet chemically bonded between the first and second sheets may be provided to induce physical restoration force of the contact switch.

In example embodiments, a projection for providing restoration force of the second sheet may be formed at an adjacent position to the switching unit.

In example embodiments, the first and second sheets may be manufactured with a PET film enabling circuit printing, and the pattern may be printed with silver ink.

In example embodiments, the switching unit may include a conductive rubber providing an electric signal from a physical contact as a switching signal from a contact. Here, the conductive rubber may be formed to have a different height for each switching portion and may be switched according to a magnitude of weight or applied pressure.

In example embodiments, the connecting unit may further include an encoding IC having an input side connected to a majority number of patterns and an output side connected to a minority number of the patterns to process signals. Here, the encoding ID may be an SMD type and may be mounted on the connecting unit.

In example embodiments, the switching unit may be a pressurized carbon fiber providing an analog signal on the basis of a resistance change according to an applied pressure.

According to some example embodiments, a shoes system for walk diagnosis using the insole sheet for walk diagnosis may include shoes for walk diagnosis including a PCB installation groove having a stepped groove at one upper side of an insole to mount a system for detecting a gait, a cover attached onto the insole, a pressure detection sheet placed on the cover to generate a switching signal corresponding to a pressure applied by a walker, and a circuit unit that is installed in the PCB installation groove, is connected to a connecting unit of the pressure detection sheet, accumulates and manages signals detected from the connecting part for each time slot, supplies wireless power to the system, and wirelessly transmits data corresponding to walk information, and a charging stage including a mounting panel for mounting the shoes for walk diagnosis. Here, the charging stage may receive commercial electricity (AC) through a power line to process the commercial electricity into pulse-type output power, may transmit wireless power to the circuit unit by using an induced electromagnetic field, may receive the walk information provided from the circuit unit, and may transmit the walk information to a wire/wireless communication network.

In example embodiments, the charging stage may further include an operation display panel at one end of the charging stage. Here, the operation display panel may visually indicate whether all the shoes for walk diagnosis are mounted, power is normally supplied, charging of the shoes for walk diagnosis is completed, data communication is completed, and communication with an external server is performed.

In example embodiments, the charging stage may include a charging unit configured to receive commercial electricity (AC) and wirelessly transmit power, and an operation control unit configured to perform charging control on the basis of a charging state, to receive the walk information provided from the shoes for walk diagnosis, and to transmit the walk information to the preset server.

In example embodiments, the charging unit may include a pulse supply circuit configured to generate a pulse signal having a certain period in response to an instruction of the operation control unit, a switching circuit configured to perform switching to a signal of a certain level in response to an output signal of the pulse supply circuit, a transmitting coil configured to form an induced electric field according to a signal supplied from the switching circuit, and a load detection circuit configured to recognize a load state on the basis of a change in a voltage of the transmitting coil and provide a result of the recognition to the operation control unit.

In example embodiments, the operation control unit may include an RF receiving unit configured to receive the walk information wirelessly transmitted from the circuit unit, a data memory configured to accumulate and manage the walk information received through the RF receiving unit for each time slot, an operation module configured to manage a communication section of the RF receiving unit, perform transmission control of the walk information accumulated in the data memory according to a certain protocol, and control an operation of the pulse supply circuit according to a result of the detection of the load detection circuit, and a communication module configured to transmit the walk information to the preset server in response to a walk information transmission instruction of the operation module.

In example embodiments, the circuit unit installed in the shoes for walk diagnosis may include a charging device configured to be tuned with an induced electromagnetic field provided from the charging unit and transform electricity obtained from the electromagnetic field into a charging voltage of the battery, and a control device configured to receive a switching signal provided from the pressure detection sheet, register and manage the switching signal for each time slot, and transmit the switching signal to the RF receiving unit of the operation control unit.

In example embodiments, the charging device may include an induction coil configured to be tuned with the electromagnetic field induced from the transmitting coil and generate a certain voltage, a rectifying circuit configured to transform the voltage induced in the induction coil into a DC voltage, a constant-current circuit configured to process an output voltage of the rectifying circuit into a set rated voltage, and a charging control circuit configured to control supply of an output voltage of the constant-current circuit to the battery according to a charging state of the battery.

In example embodiments, the control device may include an encoder configured to transform a plurality of switching signals detected in the pressure detection sheet into a certain code, a control unit configured to receive an output signal of the encoder, generate real-time data, and control wireless transmission of data for each certain time unit, a memory configured to store and manage the real-time data in response to an instruction of the control unit, and an RF transmitting unit configured to wirelessly transmit the data stored in the memory to the RF receiving unit in response to a wireless transmission control command of the control unit.

In example embodiments, the RF receiving unit and the RF transmitting unit may include a short-range communication module of any one of infrared-ray communication (IrDA), Bluetooth, RFID, ZigBee, UWB, and NFC.

According to some example embodiments, a walk diagnosis service system using the shoes system for walk diagnosis may include a network system provided with a gateway for network connection between a wire/wireless internet and a mobile communication network, a walk diagnosis server configured to register and manage personal information, unique information, and walk information provided from the shoes for walk diagnosis when at least one charging stage accesses the walk diagnosis server with a unique number through a certain authentication process based on the personal information, process gait information into graphical information on the basis of the walk information, and access the wire/wireless internet in order to receive and manage diagnosis result information on the gait information, a diagnostician terminal configured to access the wire/wireless internet, store medical history information on the basis of the personal information, and provide, to the walk diagnosis server, the diagnosis result information and alarm information generated by an external medical staff on the basis of the gait information and medical history information, and a local government server configured to monitor the diagnosis result on a walker residing in a region under jurisdiction, and notify the alarm information to a related helper mobile terminal through the mobile communication network when the alarm information on the walker is generated.

In example embodiments, the personal information may be personal details including an age, a gender, a weight, and resident registration number information.

In example embodiments, the walk diagnosis server may aggregate the walk information and may process a gait of the walker into graphical information in order to provide a weight distribution, step distance, and walk time of the walker, and the diagnostician terminal may determine a health state of the walker on the basis of the gait information of the walker of the walk diagnosis server.

In example embodiments, the diagnostician terminal may perform diagnosis on the walker on the basis of a gait state, time when a gate is unsteady, time slot of maximal activity, weight change, and energy consumption amount.

Therefore, the walk diagnosis system using the shoes for walk diagnosis in accordance with example embodiments may simplify sensors for recognizing a walk and thus may remarkably reduce the cost of production. Therefore, the shoes for walk diagnosis or analysis can be widely distributed. Further, the walk diagnosis information of the old and the infirm may be continuously accumulated, the health of the old and the infirm may be diagnosed on the basis of the information, and results of the diagnosis may be notified to local governments. As a result, the heath of the old and the infirm can be continuously managed.

### BRIEF DESCRIPTION OF THE DRAWINGS

Illustrative, non-limiting example embodiments will be more clearly understood from the following detailed description taken in conjunction with the accompanying drawings.

FIGS. 1A to 1C are a perspective view or cross-sectional view illustrating an insole for a gait diagnosis in accordance with example embodiments.

FIG. 2 is an exploded perspective view illustrating a shoe to which the insole in accordance with example embodiments is applied.

FIG. 3 is a diagram illustrating a shoe system for a gait diagnosis in accordance with example embodiments.

FIG. 4 is a block diagram illustrating operations of the system of FIG. 3.

FIG. 5 is a diagram illustrating a walk diagnosis system in accordance with example embodiments.

FIG. 6 is an image of a walk diagnosis program applied in example embodiments.

FIG. 7 is a diagram illustrating conventional shoes for diagnosing a walker.

### DETAILED DESCRIPTION OF THE EMBODIMENTS

Various example embodiments will be described more fully hereinafter with reference to the accompanying drawings, in which some example embodiments are shown. The present inventive concept may, however, be embodied in many different forms and should not be construed as limited to the example embodiments set forth herein. Rather, these example embodiments are provided so that this disclosure will be thorough and complete, and will fully convey the scope of the present inventive concept to those skilled in the art. In the drawings, the sizes and relative sizes of layers and regions may be exaggerated for clarity. Like numerals refer to like elements throughout.

It will be understood that, although the terms first, second, third etc. may be used herein to describe various elements, these elements should not be limited by these terms. These terms are used to distinguish one element from another. Thus, a first element discussed below could be termed a second element without departing from the teachings of the present inventive concept. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular example embodiments only and is not intended to be limiting of the present inventive concept. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

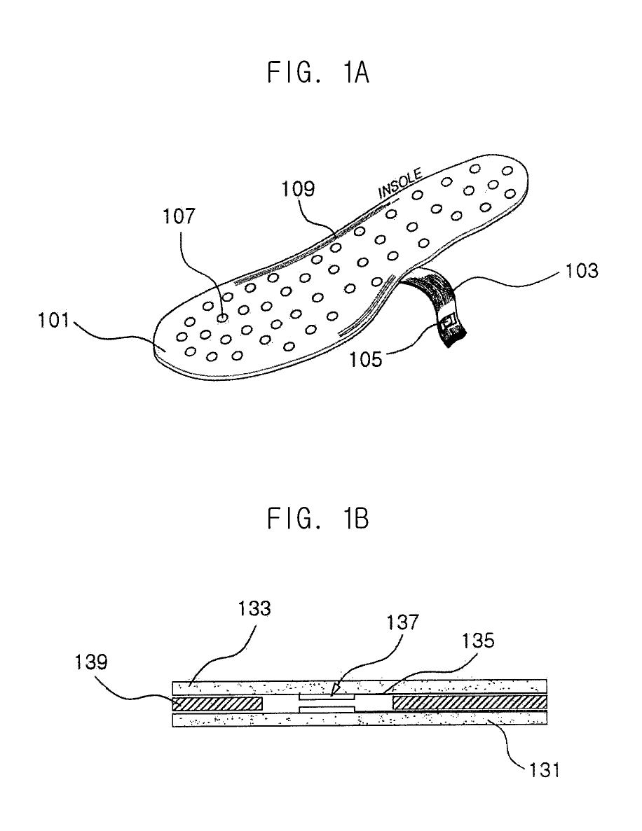

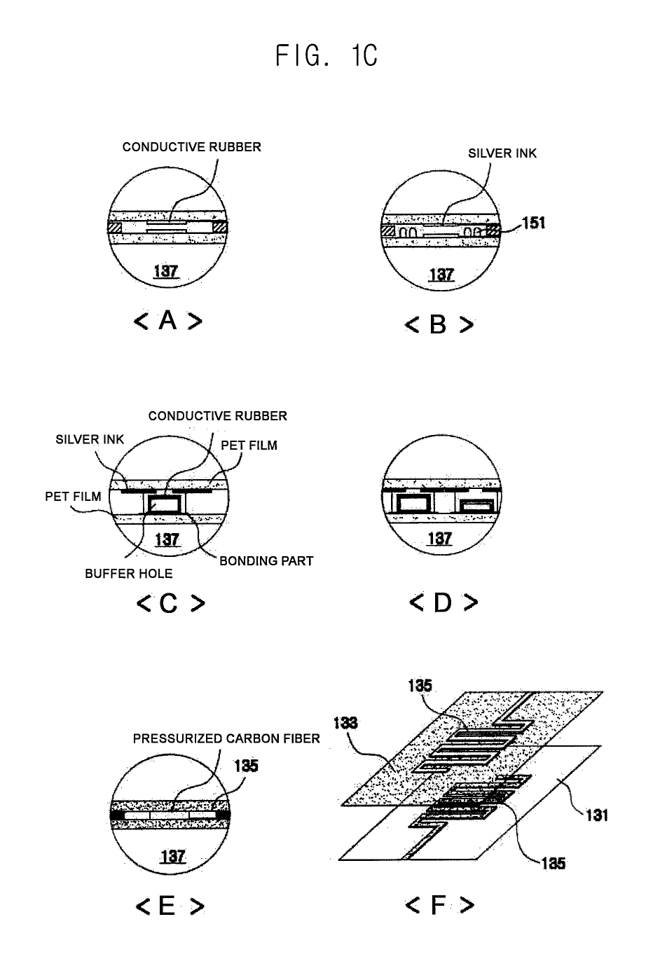

FIGS. 1A to 1C are a perspective view or cross-sectional view illustrating an insole for a gait diagnosis in accordance with example embodiments. A pressure detection sheet for collecting walk information is used as the insole of a shoe. The pressure detection sheet is described below

Referring to FIG. 1A, a pressure detection sheet 101 having a film shape is formed to have a structure corresponding to that of the insole. A plurality of switching units 107 are arranged on a surface of the pressure detection sheet 101. A circuit pattern 109 for recognizing an electrical connection between the plurality of switching units 107 is formed. A connecting unit 103 is extended to the inner side of the pressure detection sheet 101 to aggregate the circuit pattern 109.

Therefore, a signal detected in the switching unit 107 is provided through the connecting unit 103, and the switching unit 107 may provide a switching signal due to a contact point or an analog signal due to load detection. The switching signal is for providing an electric circuit signal caused by a physical contact, and the load detection provides the analog signal based on a resistance change according to an applied pressure. The pressure detection sheet 101 in accordance with example embodiments provides the switching signal or the analog signal, but, hereinafter, the two signals are not differentiated from each other and both the signals are assumed as the switching signal.

Without being limited to the operation of aggregating the circuit pattern 109, the connecting unit 103 may convert the aggregated circuit pattern to several circuit patterns. This conversion is performed through bonding of an encoding IC 105. That is, the encoding IC 105 that is an SMD type is bonded to the connecting unit 103, wherein a majority number of patterns are connected to an input side of the encoding IC 105 and a minority number of patterns are connected to an output side of the encoding IC 105. For instance, if the switching unit 107 maximally includes 512 elements, the output side of the encoding IC 105 is connected to nine patterns. Therefore, the connecting unit 103 generates bits of data for ease of signal processing, and recognizes an operation state of each switching unit 107.

FIG. 1B is a cross-sectional view of the switching unit 107 of the pressure detection sheet 101 in accordance with example embodiments. As shown, the pressure detection sheet 101 includes a first sheet 131 contacting an inner bottom of a shoe and a second sheet 133 contacting a body of a user. A contact switch 137 and a pattern 135 are formed between the first and second sheets 131 and 133 at a position corresponding to the switching unit 137. A bonding sheet 139 chemically bonded between the first and second sheets 131 and 133 is provided to induce physical restoration force of the contact switch 137.

The first and second sheets 131 and 133 allow circuit printing, and a flexible film material having high durability, such as a PET film, may be used for the first and second sheets 131 and 133. The same material may be used for the bonding sheet 139. The pattern 135 is a circuit pattern printed with silver ink.

For the contact switch 137, a conductive rubber may be used as an element having low resistance for transmitting an electric signal. That is, a rubber or silicone is mixed with carbon so as to reduce electric resistance. Instead of the conductive rubber, a pressurized carbon fiber may be used to convert a pressure applied by a walker to an analog signal. In accordance with example embodiments, a contact structure using silver ink may be included.

FIG. 1C illustrates various examples of the contact switch 137. For instance, as illustrated in (A) of FIG. 1C, the contact switch 137 may include a conductive rubber. That is, conductive rubbers are respectively bonded to the first and second sheets 131 and 133 so as to form respective patterns in the same plane shape. Therefore, in the contact switch 137 including the conductive rubbers, the two conductive rubbers contact with each other due to an externally applied pressure. Thus, the two patterns conduct electricity that may be utilized as a switching signal.

As illustrated in (B) of FIG. 1C, instead of using the conductive rubber, silver ink may be applied to opposing surfaces of the first and second sheets 131 and 133 in a predetermined pattern so as to be used as a switching means. The predetermined pattern is for minimizing an electric contact error, and may be implemented as illustrated in (F) of FIG. 1C. In the present embodiment, a projection 151 having a certain height is formed on one of the sheets 131 and 133 so as to prevent a switching error due to frequent contact. The sheets 131 and 133 may maintain elasticity for a long time and reduce a fatigue degree by virtue of materials of the sheets.

In other embodiments, as illustrated in (C) and (D) of FIG. 1C, a conductive rubber is disposed between two PET films, wherein the conductive rubber is bonded to a lower PET film, and silver ink is printed on an opposing surface of the other PET film. The printed silver ink is divided and spaced apart from each other with respect to the conductive rubber so that the two silver inks are electrically connected by the conductive rubber when the upper PET film is pressed. That is, the conductive rubber enables the two silver inks that do not contact with each other to be electrically connected to each other, so that switching is performed by the weight or pressure of a user when the user walks.

A buffer hole may be formed in a center of the conductive rubber so that the conductive rubber has a hollow structure. The buffer hole relieves the shock of the weight or pressure, thereby improving durability. As illustrated in (D) of FIG. 1C, such conductive rubbers may have different heights so that switching may be performed corresponding to the magnitude of the weight. That is, the position and magnitude of the weight is detected when a user walks, and the cost of producing the switches may be greatly reduced by virtue of such conductive rubbers.

In another embodiment, as illustrated in (E) of FIG. 1C, a pressurized carbon fiber is used as a material of the contact switch 137. A resistance of the pressurized carbon fiber is changed while the pressurized carbon fiber is pressed by an external force. Thus, the pressurized carbon fiber is bonded between the first and second sheets 131 and 133. Here, the circuit pattern may be implanted on the same plane, or may be formed on each of the two sheets. Since the internal resistance of the pressurized carbon fiber varies with the pressure applied by a walker, a pressure distribution of a body of the walker may be recognized, or the weight of the walker may be determined by averaging pressure states of a certain period of time. However, such a pressurized carbon fiber is used to detect an analog signal. An AD converting process for converting an analog signal to a digital signal is necessary for signal processing that will be described later.

As a result, as described above, the plurality of switching units 107 are formed on the pressure detection sheet 101, so that a pressure state at a predesigned position may be measured. The pressure state may be simple switching according to pressure applied by a body, or may be an analog signal based on a resistance change that is proportional to the pressure state.

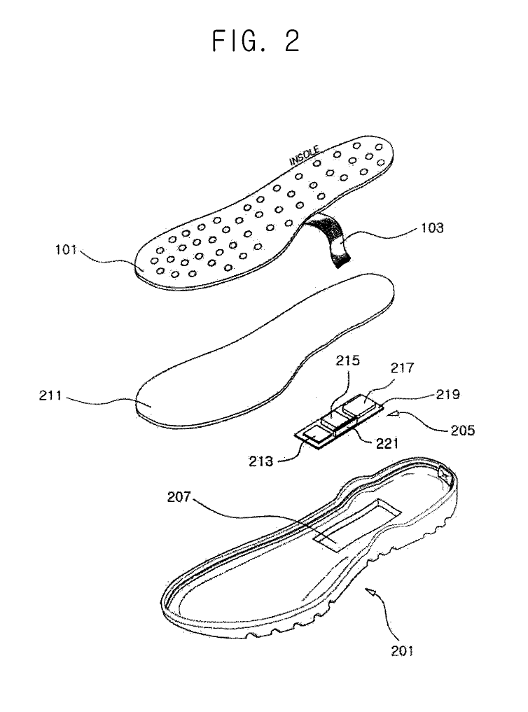

FIG. 2 is an exploded perspective view illustrating a shoe to which the insole in accordance with example embodiments is applied.

FIG. 2 illustrates a structure of the shoe using the above-described pressure detection sheet 101. FIG. 2 illustrates an insole structure for installing the pressure detection sheet of the insole for gait diagnosis in accordance with example embodiments.

As shown, a PCB installation groove 207 having a stepped groove is provided to one upper side of an insole 201 in order to place a system for detecting a gait in the PCT installation groove 207. The pressure detection sheet 101 is placed on the insole 201 and generates a switching signal corresponding to a pressured applied by a walker. A circuit unit 205 is installed in the PCB installation groove 207, accumulates and manages signals detected in the pressure detection sheet 101 for each time slot, and performs wireless power supply of a system and wireless transmission of data.

The connecting unit 103 for providing data detected from the sheet to the circuit unit 205 is provided to one side of the pressure detection sheet 101, in order for the circuit unit 205 to receive the detection data of the pressure detection sheet 101.

The circuit unit 205 includes a connecting connector 221 mounted on a printed circuit board (PCB) 219 in order to be electrically connected to the connecting unit 103, a charging device 215 that accumulates and manages in real time the data on the pressure applied by a walker and performs wireless charging by an external power supply device, and a battery 217 that charges power induced from the charging device 215 and supplies the power as system power. The circuit unit 205 may be installed with a housing having a certain shape. During a manufacturing process, the housing may be connected to the connecting unit 103, and then may be sealed or bonded so as not to disassembled or dismantled. Although it is exemplarily described that the housing is placed in the PCB installation groove 207, the housing may be fixed by a clip at the outside of the shoe. In this case, a solar cell may be applied instead of using the battery 217, or both the solar cell and the battery 217 may be applied in a dual mode. The housing may be connected to the connecting unit 103 by connecting a unified line of the connecting unit 103 through a connector.

However, in the case where the housing is designed to be placed in the PCB installation groove 207, the circuit unit 205 is placed and fixed in the PCB installation groove 207 of the insole 201, and then the connecting unit 103 of the pressure detection sheet 101 is connected to the connecting connector 221 of the circuit unit 205. As the pressure detection sheet 101 is installed on the inner side of the shoe, the connecting unit 103 passes through a side of the shoe so as to be connected to the connecting connector 221 of the circuit unit 205. The durability of the circuit unit 205 and connecting unit 103 may be improved, as necessary, through a chemical connection, or a thermal connection by means of ultrasonic waves may be possible.

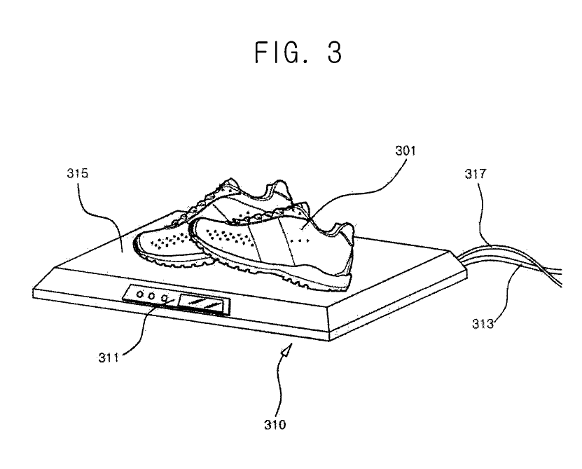

FIG. 3 is a diagram illustrating a shoes system for a gait diagnosis in accordance with example embodiments.

Referring to FIG. 3, the shoe is placed on a mounting panel 315 of a charging stage 310 that is supplied with commercial electricity (AC) through a power line 313 and wirelessly transmits power in the form of a certain pulse. The mounting panel 315 supplies power to the charging device 215 installed on the insole 201 of a shoe 301. More specifically, a wireless transmitter (not illustrated) of the charging stage 310 transmits induced electromagnetic field of a certain frequency, and the charging device 215 of the insole 201 is supplied with power through tuning with the induced electromagnetic field.

An operation display panel 311 is provided to one end of the charging stage 310. The operation display panel 311 visually indicates whether all the shoes 301 are mounted, power is normally supplied, charging of the shoes 301 is completed, data communication is completed, and communication with an external server is performed. Therefore, while the shoes 301 are mounted on the charging stage 310, the walk information accumulated during a walk is collected through wireless communication, e.g. short-range communication, so as to be transmitted to a preset server through a communication line 317 for a connection with a wire/wireless communication network. A CDMA communication module may be installed, as necessary, in the charging stage 310 so as to directly access a mobile communication network without using the communication line 317.

Since the charging of the battery 217 is performed by the charging stage 310, it is not necessary for a user to additionally manage the shoes 301. Hereinafter, functions of the charging stage 310 and shoe 301 in accordance with example embodiments will be described in detail with reference to the accompanying drawings.

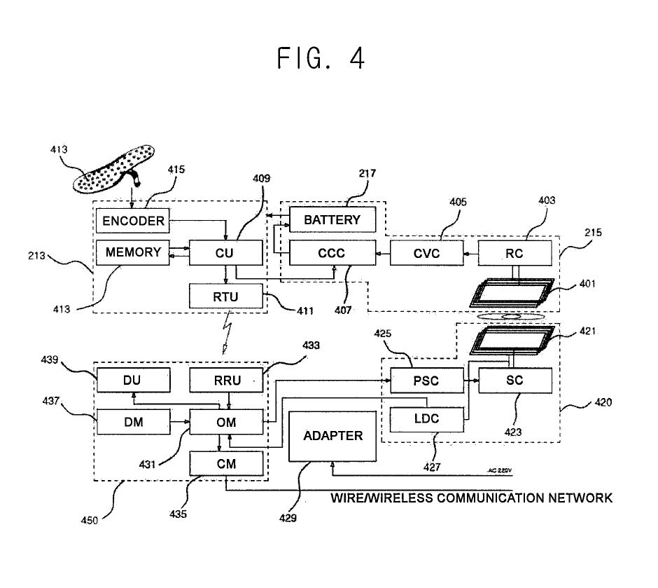

FIG. 4 is a block diagram illustrating operations of the system of FIG. 3.

FIG. 4 is a block diagram for describing the functions of the charging stage 310 and shoe 301. To be easily understood, these elements are illustrated in a single diagram. To smoothly describe the operations, an operation of the charging stage 310 will be described first.

As shown, the charging stage 310 includes a charging unit 420 that is supplied with commercial electricity (AC) to wirelessly transmit power and an operation control unit 450 that performs charging control on the basis of a charging state, receives the walk information provided from the shoe 310, and then transmits the walk information to the preset server. The charging unit 420 includes a pulse supply circuit 425 that generates a pulse signal having a certain period in response to an instruction of the operation control unit 450, a switching circuit 423 that performs switching to a signal of a certain level in response to an output signal of the pulse supply circuit 425, a transmitting coil 421 that forms an induced electric field according to a signal supplied from the switching circuit 423, and a load detection circuit 427 that recognizes a load state on the basis of a change in a voltage of the transmitting coil 421 and provides a result of the recognition to the operation control unit 450.

The operation control unit 450 includes an RF receiving unit 433 that receives the walk information wirelessly transmitted from the circuit unit 205, a data memory 437 that accumulates and manages the walk information received through the RF receiving unit 433 for each time slot, an operation module 431 that manages a communication section of the RF receiving unit 433, performs transmission control of the walk information accumulated in the data memory 437 according to a certain protocol, and controls an operation of the pulse supply circuit 425 according to a result of the detection of the load detection circuit 427, and a communication module 435 that transmits the walk information to the preset server in response to a walk information transmission instruction of the operation module 431.

The circuit unit 205 installed in the shoe 301 includes a charging device 215 that is tuned with an induced electromagnetic field provided from the charging unit 420 and transforms electricity obtained from the electromagnetic field into a charging voltage of the battery 217, and a control device 213 that receives a switching signal provided from the pressure detection sheet 101, registers and manages the switching signal for each time slot, and transmits the switching signal to the RF receiving unit 433 of the operation control unit 450.

Here, the charging device 215 includes an induction coil 401 that is tuned with the electromagnetic field induced from the transmitting coil 421 to generate a certain voltage, a rectifying circuit 403 that transforms the voltage induced in the induction coil 401 into a DC voltage, a constant-current circuit 405 that regulates an output voltage of the rectifying circuit 405 into a set rated voltage, and a charging control circuit 407 that controls supply of an output voltage of the constant-current circuit 405 to the battery 217 according to a charging state of the battery 217.

The control device 213 includes an encoder 415 that transforms a plurality of switching signals detected in the pressure detection sheet 109 into a certain code, a control unit 409 that receives an output signal of the encoder 415 to generate real-time data and controls wireless transmission of data for each certain time unit, a memory 413 that stores and manages the real-time data in response to an instruction of the control unit 409, and an RF transmitting unit 411 that wirelessly transmits the data stored in the memory 413 to the RF receiving unit 433 in response to a wireless transmission control command of the control unit 409.

The RF transmitting unit 411 and the RF receiving unit 433 may be assumed as short-range wireless communication modules, e.g. Zigbee or Bluetooth modules. Low-power analog communication may be used, as necessary.

Operations in accordance with example embodiments are described as below.

A user places the shoes 301 on the charging stage 310 after using the shoes 301. The charging stage 310 is provided with commercial electricity (AC) in order to be driven, and displays a power supply state and a charging state of the shoes 301 through the display panel 311. Further, the charging stage 310 displays whether the walk information is transmitted to the external server through the communication line 317, or displays a state and result of the transmission. If necessary, the charging stage 310 may determine whether the number of the shoes 301 is two and display a result of the determination.

The charging stage 310, which is supplied with power from the power line 313, is supplied with a rated voltage through an adapter 429, wherein the rated voltage is supplied to the operation control unit 450 and the charging unit 420. The operation module 431 of the operation control unit 450 instructs the pulse supply circuit 425 to generate a pulse signal of a certain period. In response to this instruction, the pulse supply circuit 425 provides a set frequency signal to the switching circuit 423, and a switching voltage of the switching circuit 423 drives the transmitting coil 421.

The transmitting coil 421 forms an induced electromagnetic field according to a switching signal corresponding to the pulse signal, and the induced electromagnetic field is irradiated to the induction coil 401. Here, the load detection circuit 427 measures a voltage of the transmitting coil 421. When the induced electromagnetic field irradiated from the transmitting coil 421 is applied to the adjacent induction coil 401, the voltage on the transmitting coil 421 is reduced and the load detection circuit 427 recognizes this voltage change. On the contrary, when the induction coil 401 is not adjacent to the transmitting coil 421, the voltage on the transmitting coil 421 reaches a maximal value and the load detection circuit 427 notifies a voltage drop state to the operation module 431.

The voltage drop recognized in the load detection circuit 427 is different according to a magnitude of the induced electromagnetic field accommodated by the induction coil 401. This indicates that the voltage drop is differentiated when only one of a pair of the shoes 301 is placed on the mounting panel 315 and when both the shoes 301 are placed on the mounting panel 315. Therefore, the operation module 431 determines whether shoes are placed on the mounting panel 315 or whether only one of a pair of shoes is placed thereon, on the basis of a result of the detection of the load detection circuit 427. A result of this determination is notified to the display panel 311 through a display unit 439.

The charging device 215 of the circuit unit 205 installed in the shoe 301 performs power transform to a voltage induced from the transmitting coil 421 through the induction coil 401. The induction coil 401 causes an induced voltage proportional to a transmitting frequency of the transmitting coil 421, and this voltage is full-wave rectified through the rectifying circuit 403. The rectifying circuit 403 includes a bridge diode and additionally has a smoothing circuit. Therefore, the rectifying circuit 403 transforms the induced voltage supplied from the induction coil 401 into a DC voltage. Thereafter, this voltage is transformed to a rated voltage of a system through the constant-voltage circuit 405.

The charging control circuit 407 supplies a voltage outputted from the constant-voltage circuit 405 to the battery 217. Here, according to a charge amount of the battery 217, supply of a current is limited. A lithium polymer which enables a small size of battery and is capable of storing current of high capacity may be used for the battery 217. Due to characteristics of the lithium polymer, the charging control circuit 407 includes a stabilizing circuit to prevent explosion due to a charged voltage, ambient temperature, etc. When the battery 217 is charged by the charging control circuit 407, the charged voltage of the battery 217 is supplied to the control device 103.

Therefore, the control unit 409 extracts the data stored in the memory 413, i.e. the switching data provided from the pressure detection sheet 101 attached to the insole, and storage time information on the switching data. The control unit 409 sets a certain time unit to be a single packet on the basis of the time information, and then provides the packet to the RF transmitting unit 411. For instance, the control unit 409 may transmit the walk information of a one-hour unit. As a matter of course, the control unit 409 may transmit information of a one-day unit or time information for each date as necessary.

Although the walk information on a user may be transmitted every day, the time unit is set as described above so as to transmit the data at the same time when the battery 217 is charged, e.g. every two or three days, according to a use amount of the battery. That is, the data corresponding to the walk information are transmitted in consideration of convenience of the user. Therefore, in accordance with example embodiments, it is not necessary to define a period of walk information collection. Thus, any definition of the period may not be outside the scope of example embodiments.

As described above, the RF transmitting unit 411 may be configured with a short-range wireless communication module. The short-range wireless communication module may include an infrared-ray communication (IrDA) module, a Bluetooth module, an RFID module, and a ZigBee module, and any one of communication schemes of UWB and NFC may be applied. Therefore, a plurality of short-range communication modules may also be used in the RF receiving unit 433 corresponding to the RF transmitting unit 411, and the operation module 431 receives the walk information from the RF receiving unit 433. The operation module 431 stores the received data in the data memory 437 to manage the data, and transmits the data in a certain unit of packet through the communication module 435.

As described above, the walk information generated in the circuit unit 205 in the shoe 301 is received by the charging stage 310, and then is provided to the external server through the communication line 317. Hereinafter, a walk information management system in accordance with example embodiments will be described in detail with reference to the accompanying drawings.

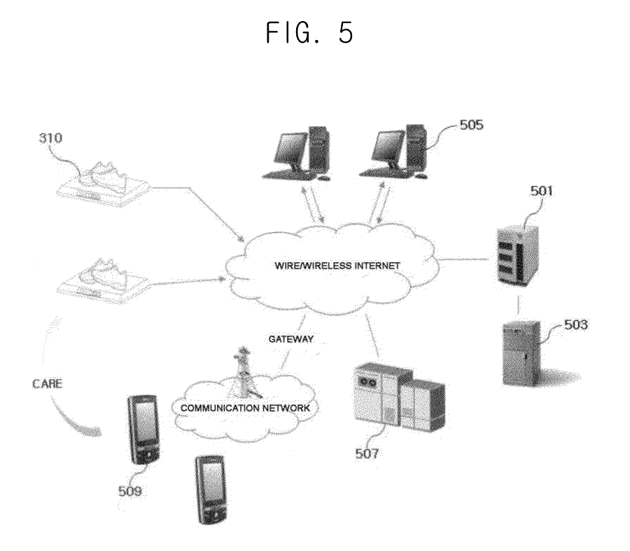

FIG. 5 is a diagram illustrating the walk information management system in accordance with example embodiments.

Referring to FIG. 5, a network system provided with a gateway for a network connection between a wire/wireless internet and a mobile communication network is formed, wherein a plurality of charging stages 310 are connected to the wire/wireless internet. Each charging stage 310 may have a unique number, and a user provides a unique number and personal information used to diagnose a gait of the user.

The unique number and personal information are registered through membership enrollment. To this end, the wire/wireless internet is connected to a walk diagnosis server 503. The walk diagnosis server 503 aggregates and manages walk information including the unique number of a shoe and the person information, stores medical history information on the basis of the personal information, and manages diagnosis result information and alarm information generated, on the basis of the walk information and medical history information, by an external medical staff. The wire/wireless internet is connected to a diagnostician terminal 505 of the medical staff, and the diagnostician terminal 505 generates and notifies a diagnosis result on the basis of the medical history information and walk information from the walk diagnosis server 503. The wire/wireless internet is connected to a local government server 507 of each region, and the local government server 507 receives the diagnosis result and personal information to recognize a health state of a walker. Here, in the case of receiving the alarm information according to the diagnosis result, the local government server 507 provides, through the mobile communication network, the alarm information to a helper mobile terminal 509 adjacent to the walker.

The personal information, which relate to personal details, includes an age, gender, and weight and includes, if necessary, resident registration number information. The walk diagnosis server 503 aggregates the walk information to provide gait information of a walker in a graphic form. This information provides a weight distribution, step distance, and walk time.

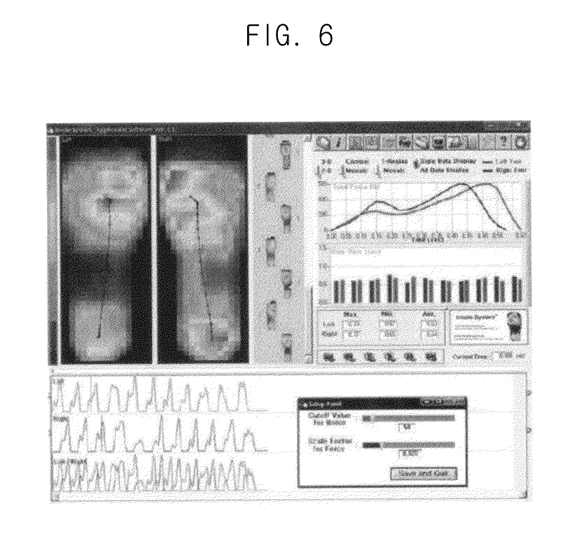

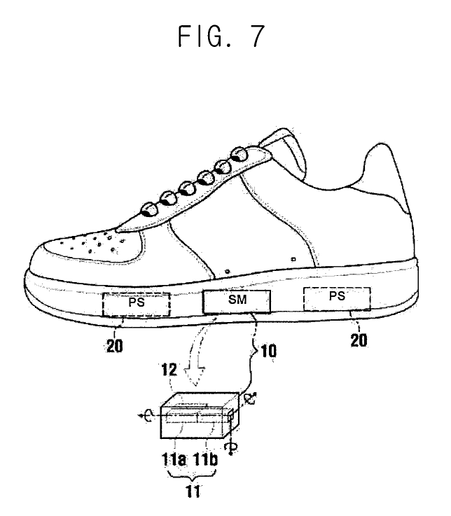

FIG. 6 is an image of a walk diagnosis program applied in example embodiments.

Referring to FIG. 6, an average of the pressure detected in the pressure detection sheet 101 for each time slot is provided in a graphic form, and information such as left-side and right-side pressure change, walk time, and step distance is provided. Therefore, the diagnostician terminal 505 determines a health state of a walker on the basis of the gait information of the walk diagnosis server 503.

That is, the diagnostician terminal 505 performs diagnosis on the walker on the basis of a gait state, time when a gate is unsteady, time slot of maximal activity, weight change, and energy consumption amount. As described above, according to a type of the pressure detection sheet 101 in accordance with example embodiments, a sheet that only generates a switching signal according to a pressure applied during a walk may be used, or a change in the pressure applied by a walker may be provided as an analog signal. The analog signal is used to measure a weight. On the basis of the measured weight, a weight change and energy consumption amount of the walker may be recognized.

Operations in accordance with example embodiments are described as below.

A user, as usual, walks by using the shoes 301 for a certain period of time. The pressure detection sheet 101 used as the insole of the shoe 301 detects a distribution of weight generated when the user walks, and stores data corresponding to the weight distribution. After the walk is finished, when the user places the shoes 301 on the charging stage 310, the charging stage 310 is connected to the wire/wireless internet through the communication line 317. The wire/wireless internet is connected to a web server 501, and the web server 501 generates a communication section with the charging stage 310.

The web server 501 is connected to the walk diagnosis server 503, and the walk diagnosis server 503 receives unique information of the shoe 301 from the charging stage 310. The walk diagnosis server 503 recognizes a membership enrollment state on the basis of the unique information, and extracts the personal information and medical history information on the basis of the unique information. The walk diagnosis server 503 receives the walk information from the charging stage 310 on the basis of the unique information of the shoe 301. The walk diagnosis server 503 stores and manages the walk information and medical history information together with the personal information so that these pieces of information are associated with each other, wherein this operation is performed for each time slot.

The walk diagnosis server 503 generates gait information on the basis of the walk information currently aggregated, and provides a result of the generation to the diagnostician terminal 505. The diagnostician terminal 505 is a terminal of the medical staff. The medical staff recognizes the gait information represented in a graphic form through the diagnostician terminal 505, and provides the diagnosis result to the walk diagnosis server 503. Here, in addition to the diagnosis result, the medical staff may generate the alarm information when a symptom of an injury is detected. The diagnosis result information is registered in the walk diagnosis server 503. Here, in the case where the alarm information is generated, the walk diagnosis server 503 notifies this information to the local government server 507 connected to the wire/wireless internet on the basis of the personal information corresponding to the alarm information.

The local government server 507 confirms whether the walker is a resident of a region under jurisdiction, and transmits the alarm information to the helper mobile terminal 509 of the region. After the helper mobile terminal 509 receives a message corresponding to the alarm information, a helper visits the walker to determine whether a physical condition of the walker is normal or to request a precise medical diagnosis. Therefore, the shoes in accordance with example embodiments aggregate and manage walk states of the walker, and determine whether there is a physical change of the walker on the basis of the a result of the management, so that a helper resolves a dangerous situation.

As described above, the shoes for walk diagnosis in accordance with example embodiments precisely recognize gaits of the old and the infirm and perform gait diagnosis on the basis of the recognition. Therefore, abnormal physical conditions of the old and the infirm may be initially detected and notified, thereby improving the health of people. Therefore, industrial applicability of such shoes is high. Moreover, the structure of the pressure detection sheet in accordance with example embodiments is simple, and thus the cost for production is remarkably reduced. Therefore, shoes for walk diagnosis may be widely distributed. Thus, industrial applicability of such a sheet is high.

The foregoing is illustrative of example embodiments and is not to be construed as limiting thereof. Although a few example embodiments have been described, those skilled in the art will readily appreciate that many modifications are possible in the exemplary embodiments without materially departing from the novel teachings and advantages of the present inventive concepts. Accordingly, all such modifications are intended to be included within the scope of the present inventive concepts as defined in the claims. Therefore, it is to be understood that the foregoing is illustrative of various example embodiments and is not to be construed as limited to the specific example embodiments disclosed, and that modifications to the disclosed example embodiments, as well as other example embodiments, are intended to be included within the scope of the appended claims.

1. An insole sheet for walk diagnosis for use in shoes for collecting walk information of a walker, wherein a pressure detection sheet having a film shape is formed to have a structure corresponding to the insole, a plurality of switching units are arranged on a surface of the pressure detection sheet, a circuit pattern for recognizing an electrical connection between the plurality of switching units is formed, and a connecting unit is extended to an inner side of the pressure detection sheet to aggregate the circuit pattern.

2. The insole sheet for walk diagnosis of claim 1, wherein the pressure detection sheet includes a first sheet contacting an inner bottom of the shoe and a second sheet contacting a body of a user, wherein a contact switch and a pattern are formed between the first and second sheets at a position corresponding to the switching unit, and a bonding sheet chemically bonded between the first and second sheets is provided to induce physical restoration force of the contact switch.

3. The insole sheet for walk diagnosis of claim 2, wherein a projection for providing restoration force of the second sheet is formed at an adjacent position to the switching unit.

4. The insole sheet for walk diagnosis of claim 2, wherein the first and second sheets are manufactured with a PET film enabling circuit printing, and the pattern is printed with silver ink.

5. The insole sheet for walk diagnosis of claim 1, wherein the switching unit includes a conductive rubber providing an electric signal from a physical contact as a switching signal from a contact, wherein the conductive rubber is formed to have a different height for each switching portion and is switched according to a magnitude of weight or applied pressure.

6. The insole sheet for walk diagnosis of claim 5, wherein the connecting unit further includes an encoding IC having an input side connected to a majority number of patterns and an output side connected to a minority number of the patterns to process signals, wherein the encoding ID is an SMD type and is mounted on the connecting unit.

7. The insole sheet for walk diagnosis of claim 1, wherein the switching unit is a pressurized carbon fiber providing an analog signal on the basis of a resistance change according to an applied pressure.

8. A shoes system for walk diagnosis using the insole sheet for walk diagnosis of claim 1, the shoes system for walk diagnosis comprising:
shoes for walk diagnosis including a PCB installation groove having a stepped groove at one upper side of an insole to mount a system for detecting a gait, a cover attached onto the insole, a pressure detection sheet placed on the cover to generate a switching signal corresponding to a pressure applied by a walker, and a circuit unit that is installed in the PCB installation groove, is connected to a connecting unit of the pressure detection sheet, accumulates and manages signals detected from the connecting part for each time slot, supplies wireless power to the system, and wirelessly transmits data corresponding to walk information; and
a charging stage including a mounting panel for mounting the shoes for walk diagnosis, wherein the charging stage receives commercial electricity (AC) through a power line to process the commercial electricity into pulse-type output power, transmits wireless power to the circuit unit by using an induced electromagnetic field, receives the walk information provided from the circuit unit, and transmits the walk information to a wire/wireless communication network.

9. The shoes system for walk diagnosis of claim 8, wherein the charging stage further includes an operation display panel at one end of the charging stage, wherein the operation display panel visually indicates whether all the shoes for walk diagnosis are mounted, power is normally supplied, charging of the shoes for walk diagnosis is completed, data communication is completed, and communication with an external server is performed.

10. The shoes system for walk diagnosis of claim 8, wherein the charging stage includes:
a charging unit configured to receive commercial electricity (AC) and wirelessly transmit power; and
an operation control unit configured to perform charging control on the basis of a charging state, receive the walk information provided from the shoes for walk diagnosis, and transmit the walk information to the preset server.

11. The shoes system for walk diagnosis of claim 10, wherein the charging unit includes:
a pulse supply circuit configured to generate a pulse signal having a certain period in response to an instruction of the operation control unit;
a switching circuit configured to perform switching to a signal of a certain level in response to an output signal of the pulse supply circuit;
a transmitting coil configured to form an induced electric field according to a signal supplied from the switching circuit; and
a load detection circuit configured to recognize a load state on the basis of a change in a voltage of the transmitting coil and provide a result of the recognition to the operation control unit.

12. The shoes system for walk diagnosis of claim 10, wherein the operation control unit includes:
an RF receiving unit configured to receive the walk information wirelessly transmitted from the circuit unit;
a data memory configured to accumulate and manage the walk information received through the RF receiving unit for each time slot;
an operation module configured to manage a communication section of the RF receiving unit, perform transmission control of the walk information accumulated in the data memory according to a certain protocol, and control an operation of the pulse supply circuit according to a result of the detection of the load detection circuit; and
a communication module configured to transmit the walk information to the preset server in response to a walk information transmission instruction of the operation module.

13. The shoes system for walk diagnosis of claim 8, wherein the circuit unit installed in the shoes for walk diagnosis includes:
a charging device configured to be tuned with an induced electromagnetic field provided from the charging unit and transform electricity obtained from the electromagnetic field into a charging voltage of the battery; and
a control device configured to receive a switching signal provided from the pressure detection sheet, register and manage the switching signal for each time slot, and transmit the switching signal to the RF receiving unit of the operation control unit.

14. The shoes system for walk diagnosis of claim 13, wherein the charging device includes:
an induction coil configured to be tuned with the electromagnetic field induced from the transmitting coil and generate a certain voltage;
a rectifying circuit configured to transform the voltage induced in the induction coil into a DC voltage;
a constant-current circuit configured to process an output voltage of the rectifying circuit into a set rated voltage; and
a charging control circuit configured to control supply of an output voltage of the constant-current circuit to the battery according to a charging state of the battery.

15. The shoes system for walk diagnosis of claim 13, wherein the control device includes:
an encoder configured to transform a plurality of switching signals detected in the pressure detection sheet into a certain code;
a control unit configured to receive an output signal of the encoder, generate real-time data, and control wireless transmission of data for each certain time unit;
a memory configured to store and manage the real-time data in response to an instruction of the control unit; and
an RF transmitting unit configured to wirelessly transmit the data stored in the memory to the RF receiving unit in response to a wireless transmission control command of the control unit.

16. The shoes system for walk diagnosis of claim 12, wherein the RF receiving unit and the RF transmitting unit include a short-range communication module of any one of infrared-ray communication (IrDA), Bluetooth, RFID, 5 ZigBee, UWB, and NFC.

17. A walk diagnosis service system using the shoes system for walk diagnosis of claim 8, the walk diagnosis service system comprising:
a network system provided with a gateway for network connection between a wire/wireless internet and a mobile communication network;
a walk diagnosis server configured to register and manage personal information, unique information, and walk information provided from the shoes for walk diagnosis when at least one charging stage accesses the walk diagnosis server with a unique number through a certain authentication process based on the personal information, process gait information into graphical information on the basis of the walk information, and access the wire/wireless internet in order to receive and manage diagnosis result information on the gait information;
a diagnostician terminal configured to access the wire/wireless internet, store medical history information on the basis of the personal information, and provide, to the walk diagnosis server, the diagnosis result information and alarm information generated by an external medical staff on the basis of the gait information and medical history information; and
a local government server configured to monitor the diagnosis result on a walker residing in a region under jurisdiction, and notify the alarm information to a related helper mobile terminal through the mobile communication network when the alarm information on the walker is generated.

18. The walk diagnosis service system of claim 17, wherein the personal information is personal details including an age, a gender, a weight, and resident registration number information.

19. The walk diagnosis service system of claim 17, wherein
the walk diagnosis server aggregates the walk information and processes a gait of the walker into graphical information in order to provide a weight distribution, step distance, and walk time of the walker; and
the diagnostician terminal determines a health state of the walker on the basis of the gait information of the walker of the walk diagnosis server.

20. The walk diagnosis service system of claim 19, wherein the diagnostician terminal performs diagnosis on the walker on the basis of a gait state, time when a gate is unsteady, time slot of maximal activity, weight change, and energy consumption amount.
